(19) United States
(12) Patent Application Publication
(10) Pub. No.: US 2007/0294103 A1
(43) Pub. Date: Dec. 20, 2007

(54) AUTOMATED LABORATORY TEST ORDERING AND RESULT TRACKING

(75) Inventors: AZMAT Z. AHMAD, KANSAS CITY, MO (US); ADAM C. LASKEY, FAIRWAY, KS (US); BRIAN J. STREICH, KANSAS CITY, MO (US); MARIA L. STECKLEIN, KANSAS CITY, MO (US); KATHY R. HILL, LENEXA, KS (US); TROY L. STOGSDILL, BLUE SPRINGS, MO (US)

Correspondence Address: SHOOK, HARDY & BACON L.L.P.;Intellectual Property Department, 2555 GRAND BOULEVARD, KANSAS CITY, MO, 64108-2613, US

(73) Assignee: CERNER INNOVATION, INC., OVERLAND PARK, KS (US)

(21) Appl. No.: 11/424,187

(22) Filed: Jun. 14, 2006

(51) Int. Cl. G06Q 10/00; A61B 5/00

(52) U.S. Cl. 705/2; 600/300

(57) ABSTRACT

Computerized systems and methods are provided for coordinating clinical laboratory testing from initial order entry to results notification. A laboratory testing manager provides a centralized conduit for interfacing entities placing orders for laboratory testing and laboratories performing testing. Entities, such as healthcare providers, patients, and the like, may access the laboratory testing manager to enter orders for laboratory testing. The laboratory testing manager may determine an eligible laboratory to perform the laboratory testing based on information provided in each order, such as the type of testing requested and insurance coverage information. After a laboratory performs testing specified in an order, the laboratory may enter the testing results, which are routed to recipients indicated for the results.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND

Clinical laboratory testing plays a key role in many facets of the healthcare process. Many of the decisions made by healthcare providers regarding patient care, from initial diagnosis through treatment and ultimate prognosis, are dictated by laboratory testing. For example, approximately 80% of all diagnoses are associated with a laboratory test. Given the crucial and prevalent role of clinical laboratory testing in the medical arena, it is imperative that the laboratory testing process provide results to healthcare providers in a timely and efficient manner.

While laboratory testing may sometimes be performed at the point-of-care, such as at a physician office or hospital, in many cases, the laboratory located at the same facility as the point-of-care may not be capable of performing a type of test required or the point-of-care location may not maintain a laboratory. In such cases, physical specimens collected from patients must be sent to a distant laboratory for testing. Currently, requesting clinical laboratory testing from distant laboratories and disseminating results are often part of a manual process, which is tedious, inefficient, and error prone. For example, in the context of a physician office requesting laboratory testing for a patient, it is often difficult for the physician office to determine an appropriate laboratory to perform the testing. Typically, the physician office will maintain information, such as the testing capabilities for each of a number of laboratories and records indicating the insurance each laboratory accepts. Accordingly, personnel at the physician office must manually review the information and determine an appropriate laboratory. This approach is prone to errors, such as a physical specimen being sent to a laboratory that is incapable of performing the test requested and/or that does not accept the patient's insurance. Moreover, as more and more physician offices do not have phlebotomists on their staff, patients are instructed to go to a particular laboratory's draw station for the testing. If the laboratory cannot perform the test or does not accept the patient's insurance, the patient must then call the physician office or his/her insurance provider to be redirected to another laboratory.

In some cases, requests for laboratory testing may be ambiguous or incomplete from the perspective of the laboratory performing the testing. For example, if a laboratory receives a request to perform testing for hepatitis, the laboratory doesn't have sufficient information to know the specific type of hepatitis for which to test. Accordingly, the laboratory may perform the incorrect test. Alternatively, the laboratory may be required to contact the requesting party (e.g., by telephone) for clarification.

Further, current reporting and tracking of laboratory testing results is often not a seamless process. Results reporting is frequently a manual process, such as through mailing, faxing, emailing, or phoning results to the appropriate healthcare provider, providing opportunities for the miscommunication of results. If a healthcare provider does not receive results in a timely manner, the provider typically must call the laboratory to determine the status of the results. In cases in which the patient's results need to be reviewed and used for care at multiple healthcare providers, it is typically the burden of the patient to carry the results from institution to institution.

Currently, some larger reference laboratories provide proprietary software and/or hardware for entering orders for laboratory testing and for accessing results. In addition, some electronic medical record systems may interface with laboratories for order entry and result viewing. However, the software and/or hardware provided by each laboratory is specific to that particular laboratory. Likewise, for electronic medical record systems, individual interfaces are required for each laboratory. Moreover, no automated process for determining an appropriate laboratory and coordinating orders and results is currently provided via such software, hardware, and interface systems.

### BRIEF SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present invention relate to providing a central mechanism for coordinating laboratory testing orders and routing of results to recipients. Accordingly, in one aspect, an embodiment of the present invention is directed to a method in a clinical computing environment for coordinating laboratory testing. The method includes receiving an order for the laboratory testing. The method also includes determining a laboratory to perform the laboratory testing. The method further includes receiving at least one of laboratory testing results and a pointer to laboratory testing results. The method still further includes providing the at least one of the laboratory testing results and the pointer to the laboratory testing results to one or more recipients.

In another aspect of the invention, an embodiment is directed to a system in a clinical computing environment for coordinating laboratory testing. The system includes an order entry component, a laboratory determining component, a laboratory testing results receiving component, and laboratory testing results routing component. The order entry component is capable of receiving an order for the laboratory testing. The laboratory determining component is capable of determining a laboratory to perform the laboratory testing. The laboratory testing results receiving component is capable of receiving at least one of laboratory testing results and a pointer to laboratory testing results. The laboratory testing results routing component is capable of providing the at least one of the laboratory testing results and the pointer to the laboratory testing results to one or more recipients.

In yet another aspect, an embodiment of the present invention relates to a method of processing an order for laboratory testing. The method includes enabling a first user to enter the order for laboratory testing. The method also includes determining one or more eligible laboratories to perform the laboratory testing based at least in part on information provided in the order for laboratory testing. Additionally, the method includes selecting one of the one or more eligible laboratories as a selected laboratory to perform the laboratory testing. The method further includes communicating the order for laboratory testing to the selected laboratory. The method also includes enabling a second user associated with the selected laboratory to provide at least one of laboratory testing results and a pointer to laboratory testing results. The method still further includes associating the at least one of the laboratory testing results and the pointer to the laboratory testing results with the order for laboratory testing and enabling a recipient to access the laboratory testing results.

### BRIEF DESCRIPTION OF THE SEVERAL VIEWS OF THE DRAWINGS

The present invention is described in detail below with reference to the attached drawing figures, wherein:

FIG. 1 is a block diagram of an exemplary computing environment suitable for use in implementing the present invention;

FIG. 2 is a block diagram showing an exemplary architecture for coordinating laboratory testing order entry and routing laboratory testing results in accordance with an embodiment of the present invention; and

FIG. 3 is a flow diagram showing a method for coordinating laboratory testing in accordance with an embodiment of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different components of methods employed, the terms should not be interpreted as implying any particular order among or between various steps herein disclosed unless and except when the order of individual steps is explicitly described.

Embodiments of the present invention provide computerized methods and systems for coordinating laboratory testing from a centralized laboratory testing manager. The laboratory testing manager provides a centralized conduit for communication of laboratory testing orders and results among entities requesting laboratory testing (e.g., hospitals, physician offices, patients, etc.), laboratories performing the testing, and intended recipient of testing results. Orders for laboratory testing may be entered in the laboratory testing manager, which may determine an eligible laboratory (e.g., from a multitude of unrelated laboratories) to perform the testing for each order. After a laboratory performs testing for an order, the results may be entered and routed to indicated recipients of the laboratory testing results.

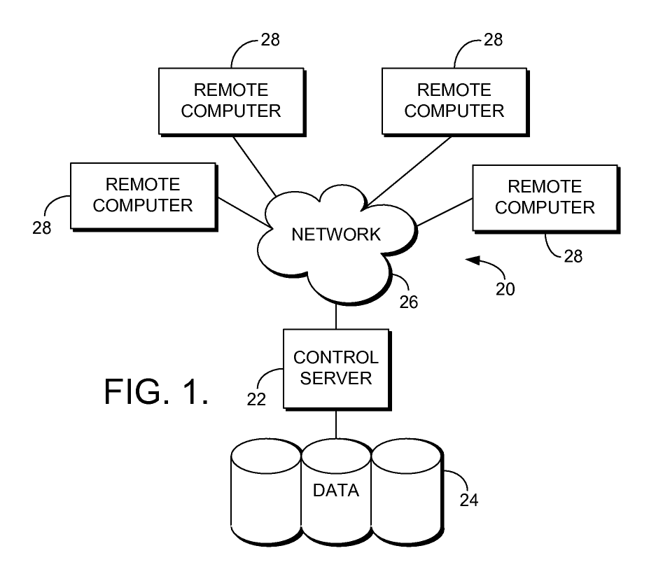

Referring to the drawings in general, and initially to FIG. 1 in particular, an exemplary computing system environment, for instance, a medical information computing system, on which embodiments of the present invention may be implemented is illustrated and designated generally as reference numeral 20. It will be understood and appreciated by those of ordinary skill in the art that the illustrated medical information computing system environment 20 is merely an example of one suitable computing environment and is not intended to suggest any limitation as to the scope of use or functionality of the invention. Neither should the medical information computing system environment 20 be interpreted as having any dependency or requirement relating to any single component or combination of components illustrated therein.

The present invention may be operational with numerous other general purpose or special purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be described in the general context of computer-executable instructions, such as program modules, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in local and/or remote computer storage media including, by way of example only, memory storage devices.

With continued reference to FIG. 1, the exemplary medical information computing system environment 20 includes a general purpose computing device in the form of a server 22. Components of the server 22 may include, without limitation, a processing unit, internal system memory, and a suitable system bus for coupling various system components, including database cluster 24, with the server 22. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The server 22 typically includes, or has access to, a variety of computer readable media, for instance, database cluster 24. Computer readable media can be any available media that may be accessed by server 22, and includes volatile and nonvolatile media, as well as removable and non-removable media. By way of example, and not limitation, computer readable media may include computer storage media and communication media. Computer storage media may include, without limitation, volatile and nonvolatile media, as well as removable and nonremovable media implemented in any method or technology for storage of information, such as computer readable instructions, data structures, program modules, or other data. In this regard, computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the server 22. Communication media typically embodies computer readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, RF, infrared, and other wireless media. Combinations of any of the above also may be included within the scope of computer readable media.

The computer storage media discussed above and illustrated in FIG. 1, including database cluster 24, provide storage of computer readable instructions, data structures, program modules, and other data for the server 22.

The server 22 may operate in a computer network 26 using logical connections to one or more remote computers 28. Remote computers 28 may be located at a variety of locations in a medical or research environment, for example, but not limited to, clinical laboratories, hospitals and other inpatient settings, veterinary environments, ambulatory settings, medical billing and financial offices, hospital administration settings, home health care environments, and clinicians' offices. Clinicians may include, but are not limited to, a treating physician or physicians, specialists such as surgeons, radiologists, cardiologists, and oncologists, emergency medical technicians, physicians' assistants, nurse practitioners, nurses, nurses' aides, pharmacists, dieticians, microbiologists, laboratory experts, genetic counselors, researchers, veterinarians, students, and the like. The remote computers 28 may also be physically located in non-traditional medical care environments so that the entire health care community may be capable of integration on the network. The remote computers 28 may be personal computers, servers, routers, network PCs, peer devices, other common network nodes, or the like, and may include some or all of the components described above in relation to the server 22. The devices can be personal digital assistants or other like devices.

Exemplary computer networks 26 may include, without limitation, local area networks (LANs) and/or wide area networks (WANs). Such networking environments are commonplace in offices, enterprise-wide computer networks, intranets, and the Internet. When utilized in a WAN networking environment, the server 22 may include a modem or other means for establishing communications over the WAN, such as the Internet. In a networked environment, program modules or portions thereof may be stored in the server 22, in the database cluster 24, or on any of the remote computers 28. For example, and not by way of limitation, various application programs may reside on the memory associated with any one or more of the remote computers 28. It will be appreciated by those of ordinary skill in the art that the network connections shown are exemplary and other means of establishing a communications link between the computers (e.g., server 22 and remote computers 28) may be utilized.

In operation, a user may enter commands and information into the server 22 or convey the commands and information to the server 22 via one or more of the remote computers 28 through input devices, such as a keyboard, a pointing device (commonly referred to as a mouse), a trackball, or a touch pad. Other input devices may include, without limitation, microphones, satellite dishes, scanners, or the like. Commands and information may also be sent directly from a remote healthcare device to the server 22. In addition to a monitor, the server 22 and/or remote computers 28 may include other peripheral output devices, such as speakers and a printer.

Although many other internal components of the server 22 and the remote computers 28 are not shown, those of ordinary skill in the art will appreciate that such components and their interconnection are well known. Accordingly, additional details concerning the internal construction of the server 22 and the remote computers 28 are not further disclosed herein.

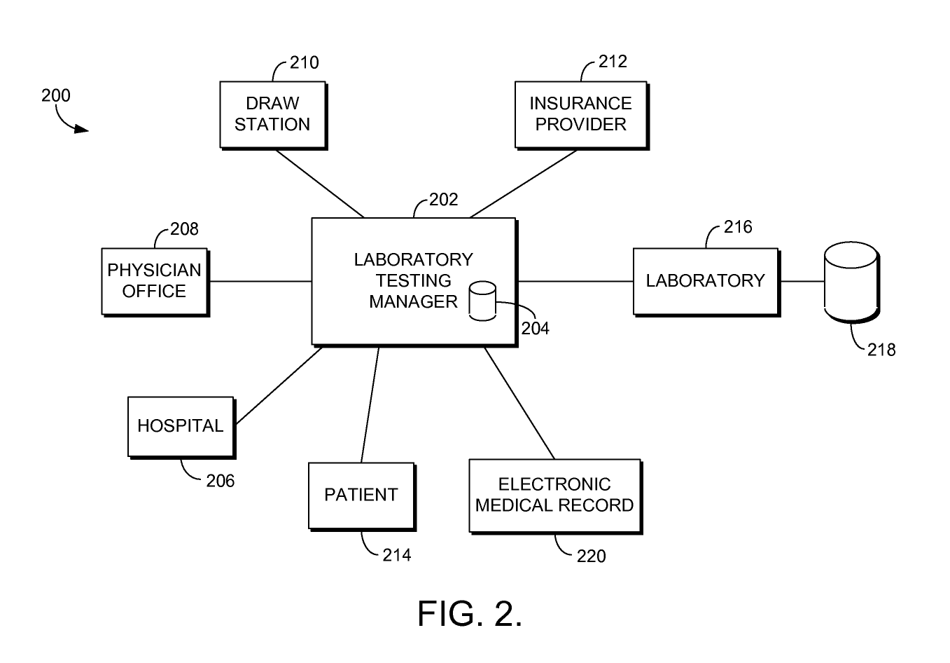

Referring now to FIG. 2, a block diagram is provided illustrating an exemplary architecture 200 for providing a centralized system for entering orders for laboratory testing and disseminating testing results in accordance with an embodiment of the present invention. As shown in FIG. 2, a laboratory testing manager 202 may be provided to coordinate, among other things, orders for laboratory testing and routing of laboratory testing results. The laboratory testing manager 202 may act as a common interface or switch between and among multiple entities requesting laboratory testing and multiple laboratories performing the testing. As such, the laboratory testing manager 202 serves as a conduit for communication of laboratory testing orders and laboratory testing results. Healthcare providers, such as hospitals, physician offices, and the like, may place orders for laboratory testing, track the status of testing, receive results, and otherwise communicate with appropriate laboratories via the laboratory testing manager 202. In addition, laboratories may receive orders for laboratory testing and route the results to the appropriate recipients via the laboratory testing manager 202.

One or more databases, such as the database 204, may be associated with the laboratory testing manager 202, for storing a variety of information to facilitate the order entry and results routing process. For example, the database 204 may maintain information regarding the types of testing available at each participating laboratory. In addition, the database 204 may maintain insurance provider information for eligibility and coverage purposes. The details and status of orders entered into the laboratory testing manager 202 may also be maintained in the database 204. Further, the database 204 may maintain testing results and/or pointers to testing results stored at a laboratory's internal database.

As shown in FIG. 2, the laboratory testing manager 202 may be capable of communicating with a number of different entities, such as a hospital 206, a physician office 208, a draw station 210, an insurance provider 212, a patient 214, and a laboratory 216, for example, for coordinating order entry and routing laboratory testing results. It should be noted that the entities shown communicating with the laboratory testing manager 202 in FIG. 2 are provided by way of example only and are not intended to limit the scope of the present invention in any way. For example, a variety of other types of entities may communicate with the laboratory testing manager 202. In addition, although only a single hospital 206, physician office 208, draw station 210, insurance provider 212, patient 214, and laboratory 216 are shown in FIG. 2, in operation, multiple hospitals, physician offices, draw stations, insurance providers, patients, and laboratories may communicate with the laboratory testing manager 202.

Each entity may have a computing device, such as a remote computer 28 of FIG. 1, for communicating with the laboratory testing manager 202. In addition, communication between the laboratory testing manager 202 and the various entities may be via one or more networks, which may comprise one or more wide area networks (WANs) and one or more local area networks (LANs), as well as one or more public networks, such as the Internet, and one or more private networks. Further, entities may be able to access the laboratory testing manager 202 in a variety of ways within the scope of the present invention. For example, in some embodiments, an entity may have a native clinical computing system, which may interface with and be able to communicate with the laboratory testing manager 202. In other embodiments, a client application associated with the laboratory testing manager 202 may reside on an entity's computing device facilitating communication with the laboratory testing manager 202. In further embodiments, communication may simply be a web-based communication, using, for example, a web browser to access the laboratory testing manager 202 via the Internet. Any and all such variations are contemplated to be within the scope of embodiments of the present invention.

In operation, an entity, such as the hospital 204, physician office 208, or draw station 210, for example, may access the laboratory testing manager 202 to enter an order for laboratory testing. In some cases, patients, such as the patient 214, may wish to order his/her own laboratory testing and may access the laboratory testing manager 202 to enter an order (e.g., via a home computer). An order comprises a request for laboratory testing to be performed and may include a variety of information, such as the type of laboratory testing requested, an identification of a physical specimen, the patient, the place of collection of a physical specimen, the time of collection of a physical specimen, insurance provider information, and intended recipients of laboratory testing results, for example.

An order for laboratory testing may be created by a user accessing the laboratory testing manager 202 (e.g., remotely via a computing device communicating with the laboratory testing manager 202) and entering the order. Typically, the order will be entered at the place the physical specimen (e.g., a blood sample, urine sample, throat swab, etc.) is collected. For example, a physician office who collects a physical specimen from a patient will typically enter the order. However, in some cases, an order may be entered from a location different from the place of collection. For example, a physician office may enter an order for a patient, while the specimen is collected from the patient at a draw station. Any and all such variations are contemplated to be within the scope of the present invention.

In some embodiments of the present invention, the laboratory testing manager 202 may automatically select a laboratory based on information entered via an order. For instance, the laboratory testing manager 202 may determine eligible laboratories based on the type of test requested and laboratory capability information maintained, for example, in database 204. In addition, the laboratory testing manager may utilize insurance provider and eligibility information to place laboratory testing orders within a patient's insurance coverage. Insurance provider information may be stored by the laboratory testing manager 202, for example, in the database 204. In some embodiments, the laboratory testing manager 202 may communicate with insurance providers, such as the insurance provider 212, to access such information. Further, laboratory preferences may be established for users such that preferred laboratories are utilized if they are determined to be eligible. In another embodiment, the insurance eligibility may be determined prior to submission of the sample and performance of the test. A variety of other factors and data may be incorporated into the process of automatic laboratory selection by the laboratory testing manager 202 within the scope of the present invention.

In further embodiments of the present invention, users may specify a particular laboratory for testing when entering an order. In such embodiments, the laboratory testing manager 202 may be configured to determine whether a user-specified laboratory is an eligible laboratory based on factors, such as laboratory capabilities, insurance eligibility, and the like. In cases in which a specified laboratory is not an eligible laboratory, the laboratory testing manager 202 may notify the user and provide an alternative laboratory.

Some types of testing and some laboratories necessitate unique specimen collection requirements. Accordingly, in some embodiments, the laboratory testing manager 202 may maintain, for example in the database 204, unique specimen collection requirements. The laboratory testing manager 202 may access and present any unique specimen collection requirements at the time of order placement based, for example, on the type of test requested and the selected laboratory.

The laboratory testing manager 202 may further incorporate an identification system for identifying physical specimens and participating laboratories. For example, the laboratory testing manager 202 may maintain or access barcode series for laboratories. In addition, the system may assign each physical specimen a unique identification code for facilitating the association of orders, physical specimens, and laboratory testing results. In some embodiments, users may print requisitions and labels for physical specimens at the time of order entry.

The laboratory testing manager 202 may maintain an account for each user entering orders and viewing results. By doing so the laboratory testing manager 202 may group orders and results for each user. Accordingly, a user may be able to access the laboratory testing manager 202 and view the status of pending orders and review testing results that have been entered. As such, the laboratory testing manager 202 may provide one or more work queues to a user allowing the user to enter orders, track the status of pending orders, and review entered results.

After a physical specimen is collected from a patient, it may be routed to a selected laboratory, such as the laboratory 216, for testing. As indicated previously, the physical specimen may be identified by a variety of identification means, such as use of an identification number, barcode, or RFID tag, for example. Such identification allows the laboratory 216 to associate the physical specimen with the appropriate order. After receiving the physical specimen, the laboratory 216 performs the specified testing, thereby obtaining testing results for the patient. The laboratory 216 may then access the laboratory testing manager 202 (e.g., via a computing device communicating with the laboratory testing manager 202) and enter the results, associating the testing results with the appropriate order. In some cases, the laboratory 216 may maintain testing results in an associated database 218 and may provide a pointer to the results to the laboratory testing manager 202, instead of providing the actual results.

The laboratory testing manager 202 may provide a work queue to the laboratory 216, thereby allowing the laboratory 216 to perform a variety of activities with respect to laboratory testing result entry. For example, the work queue may allow the laboratory 216 to view orders that are pending testing results. In some embodiments, an alert may be provided if a testing result has not been entered for an order within a predetermined period of time. In addition, the work queue allows the laboratory 216 to enter testing results or result pointers and associate the testing results/pointers with orders.

After receiving laboratory testing results and/or pointers, the laboratory testing manager 202 may store the results and/or pointers in an associated database, such as the database 204. Additionally, the laboratory testing manager 202 allows recipients (e.g., those entities indicated in the order to receive the results) to view the laboratory testing results. Results may be communicated to recipients in a variety of ways within the scope of the present invention. For example, in some embodiments, the laboratory testing manager 202 may first provide a notification to indicated recipients that laboratory testing results are available, and recipients may then access the results. The laboratory testing manager may deliver such a notification to a recipient in any number of ways, such as, for example, via an electronic mail message, a message via a client application, a message via a recipient's native clinical computing system, or a generated voice recording. In some embodiments of the present invention, the laboratory testing manager 202 may simply deliver the results and/or result pointers to the recipients. The delivery of results to recipients may be via any number of ways within the scope of the present invention, such as for example, an electronic mail message, a client application, a recipient's native clinical computing system, a generated voice recording, or via a fax machine.

In some embodiments of the present invention, the laboratory testing manager 202 may communicate with an electronic medical record 220. As such, after receiving laboratory testing results, the laboratory testing manager 202 may populate the testing results into the electronic medical record. By way of example only and not limitation, the electronic medical record 220 may comprise a community health record or personal health record.

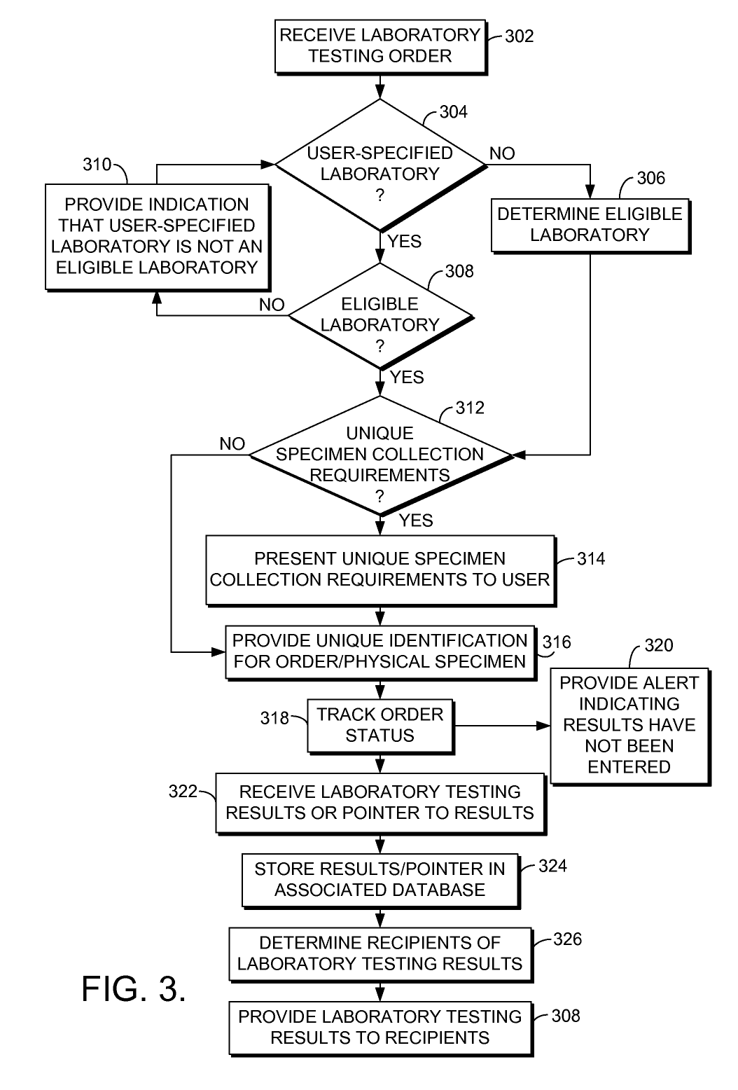

Turning now to FIG. 3, a flow diagram is provided illustrating a method 300 for coordinating laboratory testing order entry and routing of testing results in accordance with an embodiment of the present invention. Initially, as shown at block 302, a laboratory testing manager, such as the laboratory testing manager 202 of FIG. 2, receives an order for laboratory testing. For example, a physician treating a patient may wish to have a laboratory test performed for the patient. The physician or other personnel at the physician office may access the laboratory testing manager and enter the order. The order may provide a variety of information, such as the patient, the type of laboratory testing requested, insurance information, and intended recipients of results, for example.

As noted previously, in some embodiments of the present invention, the user may specify a particular laboratory the user wishes to perform the testing when entering an order. Accordingly, at block 304, a determination is made as to whether a laboratory has been specified by the user. If a laboratory is not indicated by the user, the laboratory testing manager determines an eligible laboratory to perform the requested testing, as shown at block 306. The laboratory testing manager may determine an eligible laboratory based on information provided in the order and information stored by or accessible to the laboratory testing manager, such as information regarding laboratory testing capabilities, insurance coverage, and the like. For example, based on the type of testing requested and data detailing each laboratory's testing capabilities, the laboratory testing manager may identify laboratories that may perform the requested testing. In addition, the laboratory testing manager may determine an eligible laboratory based on the patient's insurance coverage and each laboratory's insurance requirements. It should be understood that a variety of other factors and information may be used by the laboratory testing manager to determine an eligible laboratory for a particular order. In some cases, the laboratory testing manager may identify multiple eligible laboratories. Accordingly, in some embodiments, the laboratory testing manager may automatically select one of the eligible laboratories. In other embodiments, the laboratory testing manager may present the identified eligible laboratories and allow the user to select a particular laboratory.

If, at block 304, the laboratory testing manager determines that the user has specified a particular laboratory for the order, the laboratory testing manager may further determine whether the indicated laboratory is an eligible laboratory based on a variety of factors and information, such as the type of testing requested and insurance eligibility, for instance, as shown at block 308. If the selected laboratory is determined to not be an eligible laboratory, an indication may be provided to the user, as shown at block 310. The user may then specify another laboratory at block 304 or allow the system to determine an eligible laboratory at block 306 as described hereinabove.

As shown at block 312, the laboratory testing manager may determine whether there are any unique specimen collection requirements. For example, the laboratory testing manager may store a variety of specimen collection requirements in an associated database and may search the database based on, for instance, the type of testing requested and the selected laboratory. If there are any unique specimen collection requirements, the requirements are presented to the user, as shown at block 314.

In some embodiments, the laboratory testing manager may provide a unique identification for associating the order, the physical specimen, and testing results, as shown at block 316. In addition, the laboratory testing manager may provide the ability for a user to print a requisition and a label for the physical specimen. The physical specimen may then be transferred to the laboratory for the appropriate testing to be performed. As shown at block 318, the laboratory testing manager may facilitate the tracking of the status of the order. For example, the physician who ordered the laboratory testing may access the laboratory testing manager and track the status of the order, such as receipt of the physical specimen at the laboratory. In some cases, the laboratory testing manager may be configured to provide an alert if laboratory testing results are not received within a predetermined period of time after an order is entered or after the laboratory receives the physical specimen, as shown at block 320. The alert may be delivered to a variety of entities within the scope of the present invention, such as, for instance, the laboratory performing the testing and recipients of the results indicated by the order.

After the laboratory completes the testing process, the laboratory may access the laboratory testing manager and enter the testing results or a pointer to the results stored in the laboratory's internal database, as shown at block 322. The laboratory testing manager stores the results or pointer in an associated database, as shown at block 324. The results or pointer may typically be associated with the order for the laboratory testing. The laboratory testing manager may then determine recipients of the laboratory testing results, based on, for instance, information entered by the order, as shown at block 326. The laboratory testing manager may then provide the results to the recipients, as shown at block 328. Laboratory testing results may be provided to the recipients in a variety of ways within the scope of the present invention. For example, in some embodiments, the laboratory testing manager may communicate a notification of the availability of the results to the recipients. For instance, the laboratory testing manager may send an e-mail to a recipient or provide a message in the inbox within a recipient's native clinical computing system, indicating that the laboratory testing results are available. A recipient may then access the laboratory testing manager to view the results. In other embodiments, the laboratory testing manager may simply communicate the results or pointer to the recipients. In some cases, the initial recipient may review the results and use the system and method of the present invention to route the results or pointer to other recipients. For example, a clinician may wish to review routine results and route the results (or pointer) to a patient.

As can be understood, embodiments of the present invention provide a centralized mechanism for networking entities wishing to order laboratory testing with laboratories capable of performing the testing. As such, embodiments of the present invention provide a conduit for the communication of laboratory testing orders and results. The present invention has been described in relation to particular embodiments, which are intended in all respects to be illustrative rather than restrictive. Alternative embodiments will become apparent to those of ordinary skill in the art to which the present invention pertains without departing from its scope.

From the foregoing, it will be seen that this invention is one well adapted to attain all the ends and objects set forth above, together with other advantages which are obvious and inherent to the system and method. It will be understood that certain features and subcombinations are of utility and may be employed without reference to other features and subcombinations. This is contemplated and within the scope of the claims.

What is claimed is:

1. A method in a clinical computing environment for coordinating laboratory testing, the method comprising:
receiving an order for the laboratory testing;
determining a laboratory to perform the laboratory testing;
receiving at least one of laboratory testing results and a pointer to laboratory testing results; and
providing the at least one of the laboratory testing results and the pointer to the laboratory testing results to one or more recipients.

2. The method of claim 1, wherein the order comprises information associated with at least one of a type of laboratory testing, an identification of a physical specimen, a patient, a place of collection of a physical specimen, a time of collection of a physical specimen, insurance provider information, and a recipient of the laboratory testing results.

3. The method of claim 1, further comprising communicating the order to the laboratory determined to perform the laboratory testing.

4. The method of claim 1, further comprising tracking a status of the order for laboratory testing.

5. The method of claim 1, wherein determining a laboratory to perform the laboratory testing comprises:
receiving an indication of a user-specified laboratory;
determining whether the user-specified laboratory is an eligible laboratory; and
if the user-specified laboratory is an eligible laboratory, setting the user-specified laboratory as the laboratory to perform the laboratory testing.

6. The method of claim 1, wherein determining a laboratory to perform the laboratory testing comprises determining one or more eligible laboratories.

7. The method of claim 6, wherein determining one or more eligible laboratories comprises comparing information from the order against eligibility information.

8. The method of claim 7, wherein the eligibility information comprises at least one of information associated with laboratory testing capability, and laboratory insurance eligibility information.

9. The method of claim 6, wherein determining one or more eligible laboratories comprises determining one or more laboratories that are capable of performing a type of laboratory testing indicated in the order.

10. The method of claim 6, wherein determining one or more eligible laboratories comprises determining one or more laboratories within a patient's insurance coverage.

11. The method of claim 6, wherein determining a laboratory further comprises automatically selecting one of the one or more eligible laboratories.

12. The method of claim 6, wherein determining a laboratory further comprises:
presenting the one or more eligible laboratories; and
receiving a selection of one of the one or more laboratories.

13. The method of claim 1, further comprising associating the at least one of the laboratory testing results and the pointer to the laboratory testing results with the order for laboratory testing.

14. The method of claim 13, further comprising generating an alert notification if the at least one of the laboratory testing results and the pointer to the laboratory testing results are not received within a predetermined time after receiving the order for laboratory testing.

15. The method of claim 1, wherein providing the at least one of the laboratory testing results and the pointer to the laboratory testing results to one or more recipients comprises:
providing a notification to the one or more recipients of the availability of the laboratory testing results;
receiving a request to access the laboratory testing results from at least one of the one or more recipients; and
communicating at least one of the laboratory testing results and the pointer to the laboratory testing results to the at least one of the one or more recipients.

16. The method of claim 1, further comprising populating the at least one of the laboratory testing results and the pointer to the laboratory testing results into at least one of a community health record, a personal health record, and an electronic medical record.

17. One or more computer-readable media having computer-useable instructions embodied thereon for performing the method of claim 1.

18. A computer programmed to perform the method of claim 1.

19. A system in a clinical computing environment for coordinating laboratory testing, the system comprising:
an order entry component for receiving an order for the laboratory testing;
a laboratory determining component for determining a laboratory to perform the laboratory testing;
a laboratory testing results receiving component for receiving at least one of laboratory testing results and a pointer to laboratory testing results; and
a laboratory testing results routing component for providing the at least one of the laboratory testing results and the pointer to the laboratory testing results to one or more recipients.

20. A method of processing an order for laboratory testing, the method comprising:
enabling a first user to enter the order for laboratory testing;
determining one or more eligible laboratories to perform the laboratory testing based at least in part on information provided in the order for laboratory testing;
selecting one of the one or more eligible laboratories as a selected laboratory to perform the laboratory testing;
communicating the order for laboratory testing to the selected laboratory;
enabling a second user associated with the selected laboratory to provide at least one of laboratory testing results and a pointer to laboratory testing results;
associating the at least one of the laboratory testing results and the pointer to the laboratory testing results with the order for laboratory testing; and
enabling a recipient to access the laboratory testing results.

21. The method of claim 20, wherein selecting one of the one or more eligible laboratories as a selected laboratory to perform the laboratory testing comprises at least one of:
automatically selecting one of the one or more eligible laboratories as the selected laboratory to perform the laboratory testing; and
receiving a user selection of one of the one or more eligible laboratories as the selected laboratory to perform the laboratory testing.
